(19) United States
(12) Patent Application Publication
(10) Pub. No.: US 2026/0109717 A1
(43) Pub. Date: Apr. 23, 2026

(54) PHENANTHROLINE DERIVATIVE AND METHOD FOR PRODUCING PHENANTHROLINE DERIVATIVE

(71) Applicant: Toray Industries, Inc., Chuo-ku, Tokyo (JP)

(72) Inventors: Motonobu ICHIKAWA, Otsu-shi, Shiga (JP); Hitoshi TANAKA, Otsu-shi, Shiga (JP); Tsubasa OKANO, Kamakura-shi, Kanagawa (JP); Tatsuya KANZAKI, Otsu-shi, Shiga (JP)

(73) Assignee: Toray Industries, Inc., Chuo-ku, Tokyo (JP)

(21) Appl. No.: 19/485,718

(22) PCT Filed: Mar. 25, 2025
(86) PCT No.: PCT/JP2025/011694

§ 371 Date: Nov. 19, 2025

(30) Foreign Application Priority Data
Mar. 29, 2024 (JP) 2024-056038

(51) Int. Cl. C07D 519/00; H10K 50/16; H10K 59/10; H10K 85/60

(52) U.S. Cl. CPC C07D 519/00; H10K 50/16; H10K 59/10; H10K 85/6572

(57) ABSTRACT

The purpose of the present invention is to obtain a phenanthroline derivative with which it is possible to suppress a decrease in luminous efficiency even at a higher current density than conventional phenanthroline derivatives. The present invention provides a phenanthroline derivative represented by a specific formula (1), wherein, when analyzed by high-performance liquid chromatography, the absorption intensity area of a phenanthroline derivative represented by a specific formula (A) and/or a specific formula (B) is 0.001-0.300% with respect to the absorption intensity area of a phenanthroline derivative represented by specific formula (1).

### CROSS REFERENCE TO RELATED APPLICATIONS

This is the U.S. National Phase application of PCT/JP2025/011694, filed Mar. 25, 2025, which claims priority to Japanese Patent Application No. 2024-056038, filed Mar. 29, 2024, the disclosures of each of these applications being incorporated herein by reference in their entireties for all purposes.

### FIELD OF THE INVENTION

The present invention relates to a phenanthroline derivative and a method for producing a phenanthroline derivative.

### BACKGROUND OF THE INVENTION

Phenanthroline derivatives are compounds useful as light-emitting element materials for, for example, display elements, flat panel displays, backlights, lighting, interiors, signs, billboards, electrophotographic machines, and optical signal generators. As an invention of such phenanthroline derivatives, for example, there was the invention of Patent Document 1.

The invention of Patent Document 1 is an invention of a phenanthroline derivative, in which, when analyzed by high-performance liquid chromatography (hereinafter referred to as HPLC), the total content calculated from the absorption intensity area ratio of by-products having peaks at retention times of 13 minutes to 25 minutes is 0.045% or less, and in which the generation of impurities consisting of compounds in which a linking group connecting two phenanthroline derivatives is a linking group having two to five phenylene groups bonded is suppressed.

The invention was one in which a phenanthroline derivative is produced by a production method including, in this order, performing (Step 1A) to (Step 4A) continuously; (Step 5A) a step of oxidizing a reaction product of Step 4A to obtain a crude product of the phenanthroline derivative; and (Step 6A) a step of recrystallizing and further sublimating the crude product of the phenanthroline derivative of Step 5A.

### PATENT LITERATURE

Patent Document 1: Japanese Patent Laid-open Publication No. 2023-45495

### SUMMARY OF THE INVENTION

However, although the phenanthroline derivative of the invention of Patent Document 1 has the effect of enabling the obtainment of a light-emitting element having a low driving voltage and excellent durability, in the case of a light-emitting element driven, for example, by a passive matrix, it is sometimes necessary to drive the element at a high current density of up to about 200 mA/cm², and in order to repeatedly perform such driving, it has been necessary to further improve durability.

In addition, in the method for producing a phenanthroline derivative of Patent Document 1, there is a step (Step 5A) of oxidizing a reaction product of Step 4A to obtain a crude product of a phenanthroline derivative; however, for some reason, the reaction in Step 4A may not be completed, and a lithiated reaction product of Step 3A may partially remain, and there was a concern that the remaining lithiated reaction product would be oxidized to generate new impurities. Therefore, in order to remove the new impurities and obtain a high-purity phenanthroline derivative, it was necessary to repeatedly recrystallize and further sublimate the crude product of the phenanthroline derivative (Step 6A) many times.

An object of the present invention is to obtain a phenanthroline derivative with which it is possible to suppress the generation of the new impurities so that the recrystallization and sublimation steps (Step 6A) do not need to be repeatedly performed many times, and to suppress a decrease in luminous efficiency even at a higher current density than in the conventional phenanthroline derivatives.

That is, the present invention is as follows.

[1] A phenanthroline derivative having a main component represented by the following formula (1), in which, when analyzed by high-performance liquid chromatography (HPLC), the absorption intensity area of a phenanthroline derivative represented by the following formula (A) and/or the following formula (B) is 0.001% to 0.300% with respect to the absorption intensity area of the phenanthroline derivative represented by the following formula (1).

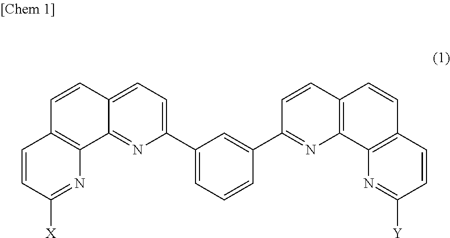

(The above X and Y each independently represent a substituted or unsubstituted aryl group.)

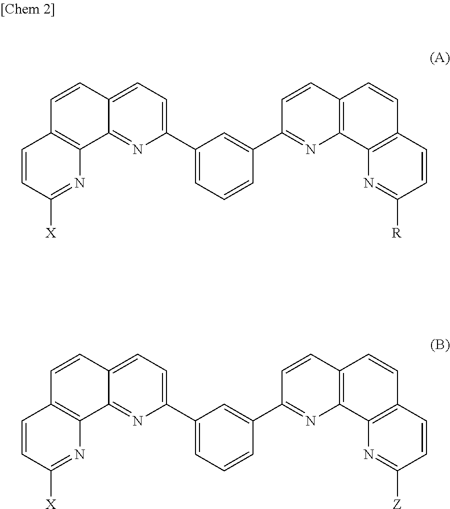

(X represents a substituted or unsubstituted aryl group; R represents any of a hydrogen atom, a halogen atom, a cyano group, a substituted or unsubstituted alkyl group having 1 to 9 carbon atoms, a cycloalkyl group, a heterocyclic group, an alkenyl group, a cycloalkenyl group, an alkynyl group, or an alkoxy group; and Z is a substituted aryl group, provided that the phenanthroline derivative represented by formula (B) has a structure different from that of the phenanthroline derivative represented by formula (1).)

[2] The phenanthroline derivative according to [1], in which the phenanthroline derivative represented by the formula (1) is a phenanthroline derivative represented by formula (3) described below.

[3] The phenanthroline derivative according to [1] or [2], in which the phenanthroline derivative represented by the formula (A) and/or the formula (B) is one or more compounds selected from the group consisting of formulas (4), (5), and (14) described below.

[4] The phenanthroline derivative according to [3], in which the phenanthroline derivative represented by the formula (A) and/or the formula (B) is two or more compounds selected from the group consisting of the formulas (4), (5), and (14).

[5] The phenanthroline derivative according to [4], in which the phenanthroline derivative represented by the formula (A) and/or the formula (B) includes at least both the compound represented by the formula (5) and the compound represented by the formula (14).

[6] The phenanthroline derivative according to any one of [1] to [5], in which, when analyzed by the high-performance liquid chromatography, the absorption intensity area of a phenanthroline derivative represented by the formula (A) and/or the formula (B) is 0.001% to 0.030% with respect to the absorption intensity area of the phenanthroline derivative represented by the formula (1).

[7] The phenanthroline derivative according to any one of [1] to [6], in which the conditions for analysis by the high-performance liquid chromatography are as follows.
Apparatus: Nexera lite system (LC-40 series) manufactured by Shimadzu Corporation
Detector: Photodiode array detector (SPD-M40) manufactured by Shimadzu Corporation
Column: A reversed-phase column packed with a filler of high-purity spherical silica gel particles chemically bonded with octyl groups
Theoretical plate number: 105,000±10,000 (N/m)
Column temperature: 45° C.
Flow rate: 1.0 mL/min
Injection amount: 10 μL
Measurement sample concentration: 5.0 mg/40 ml of tetrahydrofuran
Mobile phase: Solution A—an aqueous phosphoric acid solution having a concentration of 0.1 weight %; Solution B—an acetonitrile/tetrahydrofuran mixed solution (weight ratio 80/20)
Liquid feeding conditions: At first, a volume ratio of solution A/solution B is 55/45; after a retention time of 25 minutes, only solution B; with a linear gradient for the first 25 minutes
Analysis software: Lab Solutions manufactured by Shimadzu Corporation
Measurement wavelength: 254 nm Detection area of phenanthroline derivative: 6,000,000 or more
Minimum detectable area of compound: 60
Width (W): 1 sec
Slope(S): 1,000 μV/min
Drift (D): 300 μV/min
T.DBL (T): 1,000 min

[8] A method for producing a phenanthroline derivative including, in this order: (Step 1) a step of reacting a 1,3-dihalogenated aromatic compound with an organolithium reagent; (Step 2) a step of reacting the lithiated reaction product of Step 1 with a phenanthroline derivative of formula (6) described below; (Step 3) a step of reacting the reaction product of Step 2 with an organolithium reagent; (Step 4) a step of reacting the lithiated reaction product of Step 3 with a phenanthroline derivative represented by formula (7) described below and a phenanthroline derivative represented by formula (8) described below; (Step 5) a step of oxidizing the reaction product of Step 4 to obtain a crude product of the phenanthroline derivative; and (Step 6) a step of recrystallizing and further sublimating the crude product of the phenanthroline derivative of Step 5.

[9] A light-emitting element including: the phenanthroline derivative according to any one of [1] to [7].

[10] A display device including: the light-emitting element according to [9].

Since the phenanthroline derivative of the present invention contains a very small amount of the phenanthroline derivative represented by formula (A) and/or formula (B), there is an effect that a decrease in luminous efficiency is suppressed even at a higher current density than in the conventional phenanthroline derivatives. In addition, the method for producing a phenanthroline derivative of the present invention has an effect of being able to reduce the steps of recrystallizing and sublimating a crude product thereof, thereby greatly improving productivity.

### BRIEF DESCRIPTION OF THE DRAWINGS

FIG. 1 is an absorption spectrum obtained by HPLC analysis of a phenanthroline derivative obtained in Example 1.

FIG. 2 is a mass spectrum, including a detection peak (511.19 [M+H]+) of a compound in mass spectrometry, of a phenanthroline derivative represented by formula (A), in the absorption spectrum obtained by HPLC analysis of the phenanthroline derivative obtained in Example 1.

FIG. 3 is a mass spectrum, including a detection peak (567.25 [M+H]+) of a compound in mass spectrometry, of a phenanthroline derivative represented by formula (B), in an absorption spectrum obtained by HPLC analysis of a phenanthroline derivative obtained in Example 4.

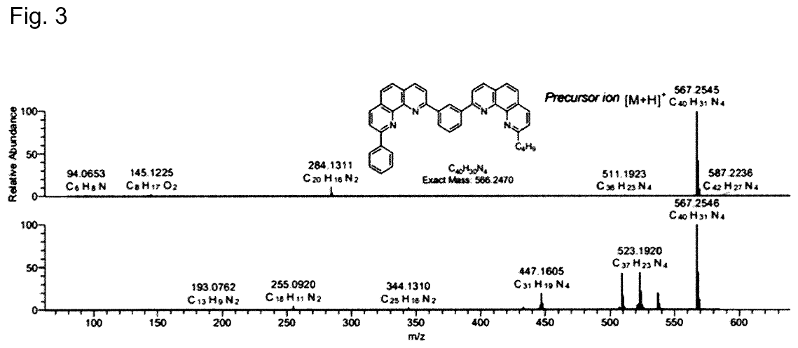

### DETAILED DESCRIPTION OF EMBODIMENTS OF THE INVENTION

Hereinafter, the present invention will be described in detail. The phenanthroline derivative of the present invention includes a compound represented by the following formula (1) as a main component. The following X and Y each independently represent a substituted or unsubstituted aryl group. The following X and Y may be the same or different. The aryl group is an aromatic hydrocarbon group obtained by removing one hydrogen atom on an aromatic ring from an aromatic hydrocarbon compound, and may be either a monocyclic ring or a condensed ring. In the present invention, even when a compound other than the structure represented by the above formula (1) is contained, it is referred to as a phenanthroline derivative.

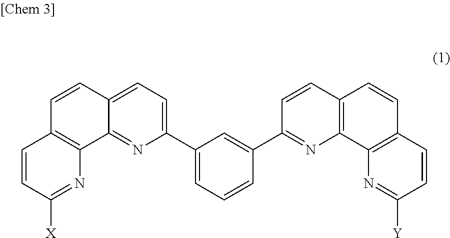

Examples of the aryl group include a phenyl group, a naphthyl group, a fluorenyl group, a benzofluorenyl group, a dibenzofluorenyl group, a phenanthryl group, an anthracenyl group, a benzophenanthryl group, a benzoanthracenyl group, a chrysenyl group, a pyrenyl group, a fluoranthenyl group, a triphenylenyl group, a benzofluoranthenyl group, a dibenzoanthracenyl group, a perylenyl group, and a helicenyl group.

The number of ring-forming carbon atoms in the aryl group is not particularly limited, but is preferably in the range of 6 or more and 40 or less, and more preferably 6 or more and 11 or less. In addition, in the case of a phenyl group, when two adjacent carbon atoms in the phenyl group each have a substituent, the substituents may form a ring structure together. The resulting group may correspond to any one or more of a “substituted phenyl group”, an “aryl group having a structure in which two or more rings are condensed”, and a “heteroaryl group having a structure in which two or more rings are condensed” depending on the structure.

Among the above aryl groups, a phenyl group, a naphthyl group, a fluorenyl group, a phenanthryl group, an anthracenyl group, a pyrenyl group, a fluoranthenyl group, and a triphenylenyl group are preferable. Furthermore, among these, a compound represented by the following formula (3), in which both X and Y are phenyl groups, is preferable in terms of low-voltage driving, durability, and the like.

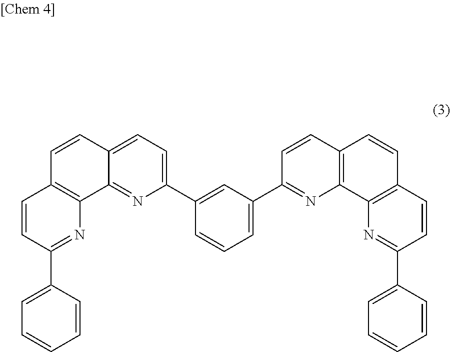 [SMILES: c1ccc(-c2ccc3ccc4ccc(-c5cccc(-c6ccc7ccc8ccc(-c9ccccc9)nc8c7n6)c5)nc4c3n2)cc1]

In the phenanthroline derivative used for organic light-emitting element materials and the like, metal ions and halogen-containing compounds derived from raw materials, by-products, solvents, and the like may be mixed into the phenanthroline derivative as a main component represented by the above formula (1). From the viewpoint of suppressing bumping during sublimation purification and further improving the characteristics of light-emitting elements such as low-voltage driving and durability, the higher the purity of the phenanthroline derivative as a main component represented by the above formula (1), the more preferable.

However, among the by-products, there are cases where, when contained in a minute amount like a dopant material or the like, they exhibit more advantageous properties than a phenanthroline derivative consisting of only the main component. Therefore, in the present invention, such useful compounds were identified, and the amount of those compounds was calculated from the absorption intensity area ratio.

Specifically, the phenanthroline derivative of the present invention is characterized in that the absorption intensity area of phenanthroline derivative(s) represented by formula (A) and/or formula (B) is 0.001% to 0.300% with respect to the absorption intensity area of the phenanthroline derivative represented by formula (1). It is considered that the phenanthroline derivative(s) represented by formula (A) and/or formula (B), when contained in a minute amount like a dopant material or the like, exhibit(s) more advantageous properties than the phenanthroline derivative consisting of only the main component, and improve(s) durability when driven at a high current density.

When the absorption intensity area of the phenanthroline derivative(s) represented by the formula (A) and/or the formula (B) exceeds 0.300% with respect to the absorption intensity area of the phenanthroline derivative represented by formula (1), it is considered that the electrical characteristics of the phenanthroline derivative represented by formula (1) are adversely affected, and the durability is conversely decreased. In addition, when the absorption intensity area of phenanthroline derivative(s) represented by the formula (A) and/or the formula (B) is less than 0.001% with respect to the absorption intensity area of the phenanthroline derivative represented by formula (1), it is considered that the effects of the present invention are difficult to obtain and sufficient durability cannot be achieved.

This durability improvement effect becomes particularly remarkable during high-temperature driving. When the light-emitting element is driven at a high temperature, it is considered that the phenanthroline derivative partially crystallizes. However, since crystallinity decreases by including the phenanthroline derivative(s) represented by formula (A) and/or formula (B), it is considered that crystals causing deterioration are less likely to form. As a further advantage of reducing crystallinity, it has also been found that there is an effect of suppressing an increase in voltage after continuous driving at a high temperature.

Here, the percentage of the absorption intensity area of the phenanthroline derivative(s) represented by formula (A) and/or formula (B) to the absorption intensity area of the phenanthroline derivative represented by formula (1) is calculated by rounding off to the third decimal place. In addition, when a plurality of phenanthroline derivatives represented by formula (A) and/or formula (B) are contained, the percentage of the total absorption intensity area of the phenanthroline derivatives represented by formula (A) and/or formula (B) to the absorption intensity area of the phenanthroline derivative represented by formula (1) is calculated.

### <High-Performance Liquid Chromatography (HPLC) Analysis>

An example of HPLC analysis conditions for the phenanthroline derivative of the present invention will be described below.
Apparatus: Nexera lite system (LC-40 series) manufactured by Shimadzu Corporation
Detector: Photodiode array detector (SPD-M40) manufactured by Shimadzu Corporation
Column: A reversed-phase column packed with a filler of high-purity spherical silica gel particles chemically bonded with octyl groups
Theoretical plate number: 105,000±10,000 (N/m)
Column temperature: 45° C.
Flow rate: 1.0 mL/min
Injection amount: 10 μL
Measurement sample concentration: 5.0 mg/40 ml of tetrahydrofuran
Mobile phase: Solution A—an aqueous phosphoric acid solution having a concentration of 0.1 weight %; Solution B—an acetonitrile/tetrahydrofuran mixed solution (weight ratio 80/20)
Liquid feeding conditions: At first, a volume ratio of solution A/solution B is 55/45; after a retention time of 25 minutes, only solution B; with a linear gradient for the first 25 minutes
Analysis software: Lab Solutions manufactured by Shimadzu Corporation
Measurement wavelength: 254 nm
Detection area of phenanthroline derivative: 6,000,000 or more
Minimum detectable area of compound: 60
Width (W): 1 sec
Slope(S): 1,000 μV/min
Drift (D): 300 μV/min
T.DBL (T): 1,000 min.

The above apparatus and detector used for the HPLC analysis enable simultaneous analysis of a main component and trace amounts of by-product compounds because of an advantage of a wide dynamic range in simultaneously analyzing a plurality of compounds with significantly different contents, such as a main component and by-products.

The above column type is a reversed-phase column corresponding to analytical validation in which polar groups on the surface of the filler are inactivated to the utmost limit. By selecting a column using a reversed-phase filler with short hydrophobic groups, in which octyl groups are chemically bonded to high-purity spherical silica gel particles, the highly hydrophobic phenanthroline derivative of the present invention can be separated and eluted in a relatively short time. In addition, the reversed-phase column corresponding to analytical validation in which polar groups on the surface of the filler are inactivated to the utmost limit is suitable for the separation and elution of aromatic organic compounds.

Furthermore, by selecting a column size having a theoretical plate number of 105,000±10,000 (N/m), which serves as an index of the packing state of the column, trace amounts of compounds can be detected with high accuracy. Examples of such a column include Mightysil RP-8GP (manufactured by Kanto Chemical Co., Inc.). It is preferable to select a column size with a length of 250 mm and an inner diameter of 4.6 mm (particle diameter: 5 μm). The above column temperature, flow rate, injection amount, and measurement sample concentration in the analysis are all general-purpose conditions.

As the mobile phase, solution A, which is an aqueous phosphoric acid solution having a concentration of 0.1 weight %, and solution B, which is an acetonitrile/tetrahydrofuran mixed solution (weight ratio 80/20), are prepared. At first, a volume ratio of solution A/solution B is 55/45; after a retention time of 25 minutes, only solution B; and during this period (the first 25 minutes), the liquid is delivered under linear gradient elution conditions.

The gradient refers to a development condition in which the composition of solution B in the mobile phase increases with the elapse of time, and the linear gradient refers to a liquid feeding condition in which the amount of solution B in the mobile phase increases at a constant rate in proportion to the retention time. By selecting such a liquid feeding condition of a linear gradient, it is possible to form peaks of by-products having long retention times into sharp shapes that are easy to detect while suppressing overlapping of a plurality of peaks.

Under the above conditions, the content of each component eluted from the column can be qualitatively and quantitatively analyzed from an ultraviolet absorption spectrum at a measurement wavelength of 254 nm.

Specifically, using Lab Solutions (manufactured by Shimadzu Corporation) as the analysis software, the conditions that allow uniform detection of minute peaks are set as follows: Width (W), representing the minimum detectable peak width, is 1 sec; Slope(S), representing the peak detection sensitivity for determining the peak start/end points, is 300 μV/min; Drift (D), representing the slope for complete peak separation (baseline separation), is 300 μV/min; and T.DBL (T), representing the specified time for changing the values of Slope and Drift, is 1,000 min.

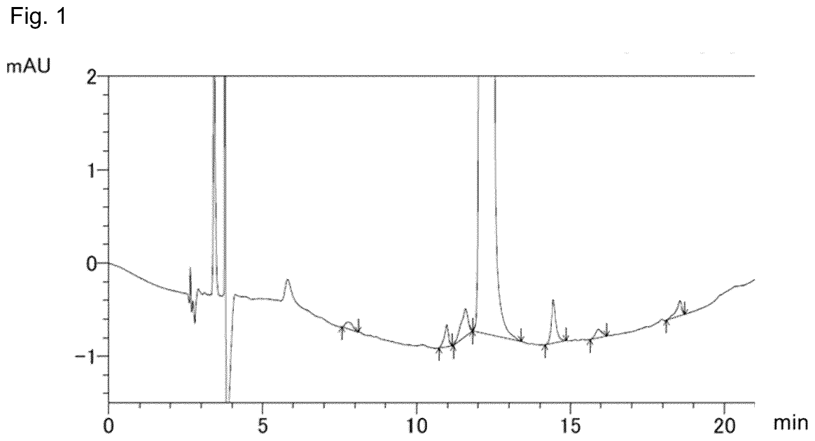

When HPLC analysis is performed under the above conditions, the peak of the phenanthroline derivative having the structure represented by the formula (1) is detected at a retention time of 12 minutes to 13 minutes. The detection area of the phenanthroline derivative is 6,000,000 or more, and a peak of the compound is detected by defining 0.001% or more of that area as a minimum compound detection area. As an example, FIG. 1 shows an absorption spectrum obtained by HPLC analysis of a phenanthroline derivative of the present invention obtained in Example 1 described below.

In addition, when HPLC analysis is performed under the above conditions, the peak of the phenanthroline derivative(s) represented by formula (A) and/or formula (B) is detected at a retention time of 7 minutes to 8 minutes.

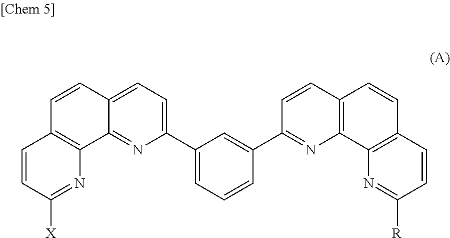

The phenanthroline derivative represented by the above formula (A) has a structure in which, among the phenanthroline derivatives represented by the above formula (1), Y is substituted with R, where R represents any of a hydrogen atom, a halogen atom, a cyano group, a substituted or unsubstituted alkyl group having 1 to 9 carbon atoms, a cycloalkyl group, a heterocyclic group, an alkenyl group, a cycloalkenyl group, an alkynyl group, or an alkoxy group.

The alkyl group represents a saturated aliphatic hydrocarbon group such as a methyl group, an ethyl group, an n-propyl group, an isopropyl group, an n-butyl group, a sec-butyl group, a tert-butyl group, or a pentyl group. The cycloalkyl group represents a saturated alicyclic hydrocarbon group such as a cyclopropyl group, a cyclohexyl group, a norbornyl group, or an adamantyl group. The alkenyl group represents an unsaturated aliphatic hydrocarbon group containing a double bond, such as a vinyl group or a butadienyl group.

The cycloalkenyl group represents an unsaturated alicyclic hydrocarbon group containing a double bond, such as a cyclopentenyl group, a cyclopentadienyl group, or a cyclohexenyl group. The alkynyl group represents an unsaturated aliphatic hydrocarbon group containing a triple bond, such as an ethynyl group. The alkoxy group represents a functional group in which an aliphatic hydrocarbon group is bonded via an ether bond, such as a methoxy group, an ethoxy group, or a propoxy group.

Among the compounds represented by the above formula (A), those in which R, which easily and smoothly reacts with the lithiated reaction product remaining in Step 4, is a hydrogen atom, a halogen atom, a cyano group, or a substituted or unsubstituted alkyl group having 1 to 9 carbon atoms are preferable. More preferred examples of the compounds include compounds represented by the following formulas (4) and (5).

The phenanthroline derivative represented by the formula (B) is a compound different from the phenanthroline derivative represented by formula (1), which is the main component, among the phenanthroline derivatives represented by the above formula (1). Among the compounds represented by the formula (B), a compound represented by formula (14) is preferable.

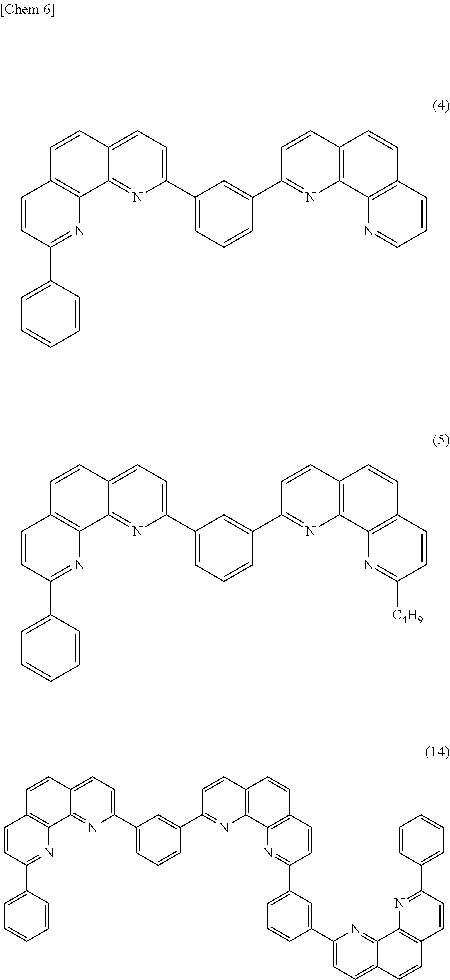

When the phenanthroline derivative(s) represented by the above formula (A) and/or the above formula (B) are present in an extremely small proportion relative to the phenanthroline derivative represented by the above formula (1), they exhibit a property of suppressing a decrease in luminous efficiency under high current density. Although the mechanism has not been clarified, it is presumed that deactivation of active species of excitons, particularly triplet excitons, generated by driving under high current density, is suppressed, and that the excitons can be continuously utilized in the light emission transition process.

The phenanthroline derivative represented by the formula (1) can be obtained, for example, by reacting a 1,3-dihalogenated aromatic compound with an organolithium reagent to form a delithiated product, allowing a phenanthroline derivative represented by the following formula (6) to act on it, subsequently reacting the reaction product with an organolithium reagent, then reacting the resulting reaction product with a phenanthroline derivative of the following formula (7), and oxidizing the product.

In addition, the phenanthroline derivative represented by the formula (A) can be obtained by allowing a phenanthroline derivative of the following formula (8) to act instead of the phenanthroline derivative of the following formula (7). The phenanthroline derivative represented by the formula (A) thus obtained is weighed in a trace amount and mixed with the phenanthroline derivative represented by the formula (1), thereby obtaining the phenanthroline derivative of the present invention.

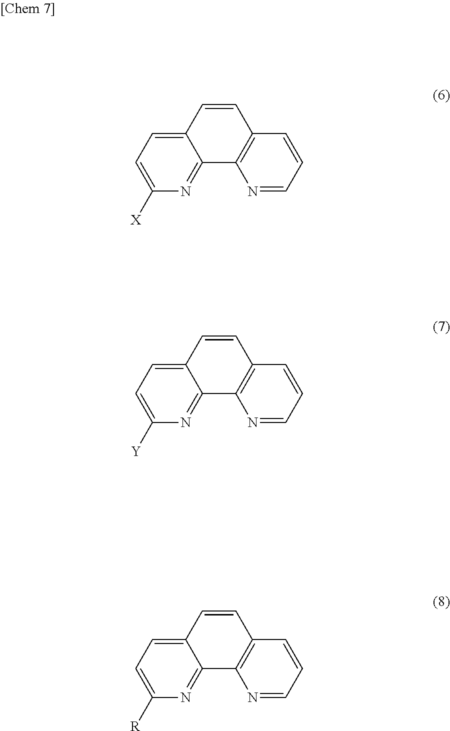

Alternatively, the following production method can also be exemplified, in which the phenanthroline derivative of the formula (1) and the phenanthroline derivative of the formula (A) are continuously and simultaneously synthesized in a series of steps. The production method includes: (Step 1) a step of reacting a 1,3-dihalogenated aromatic compound with an organolithium reagent; (Step 2) a step of reacting a lithiated reaction product of Step 1 with a phenanthroline derivative of formula (6); (Step 3) a step of reacting the reaction product of Step 2 with an organolithium reagent; and (Step 4) a step of reacting the lithiated reaction product of Step 3 with a phenanthroline derivative of formula (7) and a phenanthroline derivative of formula (8).

In this step, even when the reaction between the lithiated reaction product of Step 3 and the phenanthroline derivative of formula (7) in Step 4 is not completed for some reason and the lithiated reaction product of Step 3 remains, the remaining lithiated reaction product reacts with the phenanthroline derivative of formula (8). Therefore, almost no unreacted lithiated reaction product of Step 3 remains in the subsequent step, and the generation of new impurities is suppressed.

As a result, in (Step 5) a subsequent step of oxidizing the reaction product of Step 4 to obtain a crude product of a phenanthroline derivative, almost no by-products other than the phenanthroline derivative of formula (1) and a small amount of the phenanthroline derivative of formula (A) are produced. Furthermore, conventionally, in order to remove impurities, it was sometimes necessary to repeatedly perform (Step 6) a step of recrystallizing and further sublimating the crude product of the phenanthroline derivative of Step 5 many times; however, the number of repetitions can be significantly reduced, thereby improving the productivity of the phenanthroline derivative of the present invention.

In addition, by appropriately controlling the process of (Step 5), it is possible to produce a product containing the phenanthroline derivative of formula (1) and an appropriate trace amount of the phenanthroline derivative of formula (A). As a result, a phenanthroline derivative having an excellent property of suppressing a decrease in luminous efficiency under high current density, which is superior to that of the phenanthroline derivative of formula (1) alone, can be obtained.

All of these steps may be performed by a batch production method; however, in order to further improve productivity, (Step 1) to (Step 4) may be performed by a process of a flow synthesis method (hereinafter referred to as “flow”). That is, it is a process in which raw materials are sent from reservoirs into flow channels by a liquid-feeding pump, mixed in a mixer section, reacted in a narrow tubular reaction vessel (reactor) while flowing through the flow channels, and the resulting compounds are discharged from the flow channels.

In the flow, the diffusion rate and reaction rate of the raw materials depend on the size of the reaction vessel. Because the space for mixing raw materials is smaller than in the batch method, the raw materials are mixed accurately and quickly at a mixing ratio determined by the flow rate. Accordingly, it is possible to suppress a decrease in the reaction rate and further suppress side reactions. In addition, the flow makes it easier to maintain a uniform temperature throughout the reaction system, thereby suppressing the formation of by-products caused by side reactions due to localized temperature increases.

Examples of the pump include plunger pumps and diaphragm pumps. In addition, examples of the shape of the mixer include a T-shape and a Y-shape. The flow channel diameter (inner diameter) of the mixer is preferably 100 μm to 3 mm.

The raw materials will be described in detail below. Examples of the 1,3-dihalogenated benzene used in (Step 1) include 1,3-dibromobenzene, 1,3-diiodobenzene, and 1-bromo-3-iodobenzene. Two or more types of these may be used. Among these, 1,3-dibromobenzene is preferable.

Examples of the organolithium reagent include n-butyllithium, sec-butyllithium, and tert-butyllithium. Two or more types of these may be used. Among these, n-butyllithium is preferable.

These are preferably reacted in a solution state. Examples of the solvent include saturated hydrocarbons having 5 to 8 carbon atoms, such as pentane, hexane, heptane, octane, and cyclohexane; and ethers such as tert-butyl methyl ether, cyclopentyl methyl ether, dimethyl ether, diethyl ether, dibutyl ether, 1,4-dioxane, and tetrahydrofuran. Two or more types of these may be used. The solution concentration of each raw material is preferably 5.0 M or less, and more preferably 2.0 M or less.

The organolithium reagent is preferably used in an amount of 0.8 to 1.1 equivalents relative to 1,3-dihalogenated benzene. The flow rate (ml/min) of each raw material is preferably 1 ml/min or more, and more preferably 4 ml/min or more. The reaction temperature is preferably −20° C. to 40° C. The residence time (reaction time) in (Step 1) is preferably 0.1 to 3 seconds in consideration of the time required for halogen-lithium exchange reaction and the stability time of the lithiated reaction product. It is more preferably 2.5 seconds or less from the viewpoint of further reducing the above-described by-products.

Examples of the phenanthroline derivative of formula (6) used in (Step 2) include 2-phenyl-1,10-phenanthroline, 2-(4-hydroxyphenyl)-1,10-phenanthroline, 2-(4-methoxyphenyl)-1,10-phenanthroline, 2-(4-tert-butyl)phenyl-1,10-phenanthroline, 2-(4-tert-butyl)phenyl-1,10-phenanthroline, and 2-(3,5-di-tert-butyl-4-methoxyphenyl)-1,10-phenanthroline. It is preferable to use 0.8 to 1.1 equivalents thereof. In addition, the residence time in (Step 2) is preferably 4 to 6 seconds in consideration of the time required for the reaction of phenanthroline. The reaction temperature is preferably-20° C. to 40° C.

A preferred mode of (Step 3) is similar to that of (Step 1). Examples of the phenanthroline derivative of formula (7) used in (Step 4) include compounds similar to those of formula (6) used in (Step 2) above. When the same raw materials as those used in (Step 2) are used, a preferred mode of (Step 4) is similar to that of (Step 2).

Examples of the phenanthroline derivative of formula (8) used in (Step 4) include 1,10-phenanthroline, 2-methyl-1,10-phenanthroline, 2-ethyl-1,10-phenanthroline, 2-isopropyl-1,10-phenanthroline, 2-butyl-1,10-phenanthroline, 2-tert-butyl-1,10-phenanthroline, and 2-cyclohexyl-1,10-phenanthroline.

Examples of oxidizing agents used for the oxidation reaction in (Step 3) include manganese dioxide, nitrobenzene, chloranyl, DDQ, air, oxygen, and water. Two or more types of these may be used. The amount of the oxidizing agent can be appropriately selected depending on the oxidizing agent used, and is preferably 1 to 10 equivalents relative to formula (7).

Examples of the solvent used in the oxidation step include aromatic hydrocarbon-based solvents such as benzene, toluene, xylene, and nitrobenzene; halogenated hydrocarbon-based solvents such as dichloromethane and chloroform; ether-based solvents such as diethyl ether, tert-butyl methyl ether, cyclopentyl methyl ether, tetrahydrofuran, 1,4-dioxane, and dimethoxyethane; and N, N-dimethylformamide and N,N′-dimethylimidazolidinone (DMI). Two or more types of these may be used. The reaction temperature can be appropriately selected depending on the oxidizing agent used, and is preferably −20° C. to 60° C. The reaction time is preferably about 10 minutes to 24 hours.

Examples of solvents used for recrystallization in (Step 6) include toluene, hexane, tetrahydrofuran, dioxane, dimethoxyethane, ethanol, methanol, acetone, methyl ethyl ketone, ethyl acetate, n-butyl lactone, nitrobenzene, dichloromethane, chloroform, dimethyl sulfoxide, dimethylformamide, 1-methyl-2-pyrrolidone, 1,3-dimethyl-2-imidazolidinone, pyridine, and triethylamine. Two or more types of these may be used.

The heating temperature and crystallization temperature for recrystallization can be appropriately selected depending on the solvent used and are preferably 0° C. to 130° C. The sublimation temperature is preferably 400° C. or lower, and the degree of vacuum during sublimation purification is preferably 1.0×10⁻³ Pa or less. As described above, according to the method for producing a phenanthroline derivative of the present invention, a phenanthroline derivative in which the absorption intensity area of the phenanthroline derivative(s) represented by formula (A) and/or formula (B) is 0.001% to 0.030% with respect to the absorption intensity area of the phenanthroline derivative represented by formula (1) can be easily obtained.

### EXAMPLES

Evaluations in Examples and Comparative Examples were performed by the following methods.

### (1) Analysis by HPLC

Each phenanthroline derivative obtained in Examples and Comparative Examples was weighed in an amount of 4.0 mg into a glass container, and dissolved by adding 40 ml of tetrahydrofuran. 1 ml of the prepared solution was transferred to an HPLC vial bottle to prepare an HPLC measurement sample. Then, analysis was performed under the following conditions by HPLC. The absorption intensity areas of the phenanthroline derivative represented by formula (1) and the phenanthroline derivative(s) represented by formula (A) and/or formula (B) were calculated.
Apparatus: Nexera lite system (LC-40 series) manufactured by Shimadzu Corporation
Detector: Photodiode array detector (SPD-M40) manufactured by Shimadzu Corporation
Column type: A reversed-phase column packed with a filler of high-purity spherical silica gel particles chemically bonded with octyl groups
Theoretical plate number: 105,000±10,000 (N/m)
Column temperature: 45° C.
Flow rate: 1.0 mL/min
Injection amount: 10 μL
Measurement sample concentration: 5.0 mg/40 ml of tetrahydrofuran
Mobile phase: Solution A—an aqueous phosphoric acid solution having a concentration of 0.1 weight %; Solution B—an acetonitrile/tetrahydrofuran mixed solution (weight ratio 80/20)
Liquid feeding conditions: At first, a volume ratio of solution A/solution B is 55/45; after a retention time of 25 minutes, only solution B; with a linear gradient for the first 25 minutes
Analysis software: Lab Solutions manufactured by Shimadzu Corporation
Measurement wavelength: 254 nm
Detection area of phenanthroline derivative: 6,000,000 or more
Minimum detectable area of compound: 60
Width (W): 1 sec
Slope(S): 1,000 μV/min
Drift (D): 300 μV/min
T.DBL (T): 1,000 min.

### (2) Analysis of Structural Formula by Mass Spectrometry System

Among the phenanthroline derivatives obtained in each of Examples and Comparative Examples, the structural formulas of a compound having peaks at retention times of 12 minutes to 13 minutes and a compound having peaks at retention times of 7 minutes to 8 minutes were detected by a mass spectrometry system directly connected to an elution port of the above liquid chromatography. The conditions of mass spectrometry are as follows.
Apparatus: Orbitrap Fusio Tribrid mass spectrometer (manufactured by Thermo Scientific Inc.)
Ionization method: Electrospray ionization method
Spray voltage: Static
Positive ion: 3500 V
Measurement mode: scan mode
Vaporizer temperature: 400° C.
Ion Transfer Tube temperature: 350° C.
Sheath Gas: 60 Arb
Aux Gas: 15 Arb
Sweep Gas: 2 Arb
MS/MS: Data-Dependent MSn scan mode
Dissociation procedure: HCD
Collision Energy Mode: Stepped
Type: Normalized
HCD collision energy: 35, 65, 95%.

### (3) Driving Voltage and Durability of Light-Emitting Element

A glass substrate on which an ITO transparent electroconductive film was deposited in a thickness of 165 nm (manufactured by GEOMATEC Co., Ltd., 11Ω/□, sputtered product) was cut into 38 mm×46 mm, and etched. The resulting substrate was ultrasonically washed for 15 minutes using Semico Clean 56 (product name, manufactured by Furuuchi Chemical Corporation), and was then washed with ultra-pure water. This substrate was treated with UV-ozone for 1 hour immediately before preparation of an element, and placed in a vacuum deposition apparatus, and the apparatus was evacuated until the degree of vacuum in the apparatus was 5×10⁻⁴ Pa or less. By a resistance heating method, first, as a hole injection layer, HAT-CN6 represented by the following formula (9) was deposited to a thickness of 5 nm, and next, as a hole transport layer, HT-1 represented by the following formula (10) was deposited to a thickness of 50 nm.

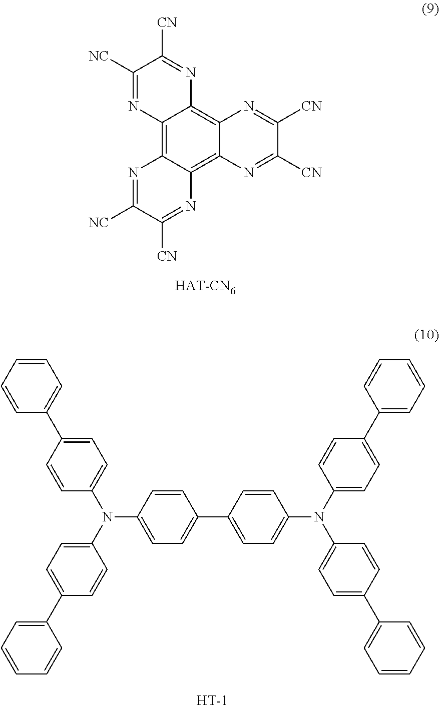

Next, as a light-emitting layer, a mixed layer of a host material H-1 represented by the following formula (11) and a dopant material D-1 represented by the following formula (12) was deposited to a thickness of 20 nm so that the doping concentration would be 5 weight. Next, as an electron transport layer, ET-1 represented by the following formula (13) was deposited to a thickness of 30 nm.

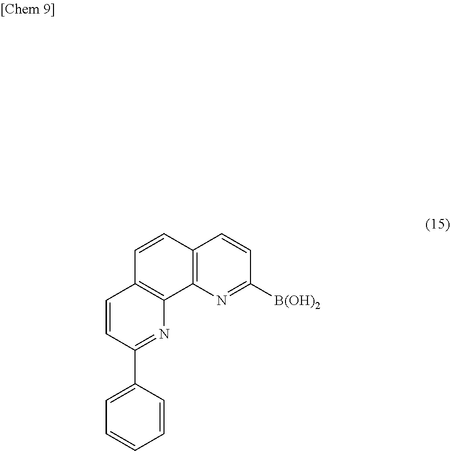 [SMILES: OB(O)c1ccc2ccc3ccc(-c4ccccc4)nc3c2n1]

Next, as an N-type charge generation layer, a mixed layer of the phenanthroline derivative obtained in each of Examples and Comparative Examples and lithium was deposited to a thickness of 10 nm so that the doping concentration of lithium would be 1 weight %. Next, as a P-type charge generation layer, HAT-CN6 was deposited to a thickness of 10 nm. Thereafter, lithium fluoride was deposited to a thickness of 0.5 nm, and aluminum was deposited to a thickness of 1000 nm to form a cathode, thereby producing a light-emitting element with a 5 mm×5 mm square.

The obtained light-emitting element was driven with a direct current at 10 mA/cm², and the initial driving voltage was measured. Further, the luminance when the element was driven with a direct current at a current density of 100 mA/cm² under conditions of a temperature of 20° C. to 30° C. was measured, and the time until the luminance decreased by 5% from the initial luminance was evaluated as durability.

### (4) Durability Evaluation of Light-Emitting Element During High-Temperature Driving

A light-emitting element was produced by the same procedure as in (3), and the luminance when the element was driven with a direct current at a current density of 100 mA/cm² under a condition of a temperature of 50° C. was measured, and the time until the luminance decreased by 10% from the initial luminance was evaluated as durability.

(5) Evaluation of Voltage Increase after High-Temperature Driving

A light-emitting element was produced by the same procedure as in (3), and after being continuously driven with a direct current for 1000 hours at a current density of 100 mA/cm² under a condition of a temperature of 50° C., the driving voltage was measured when driven with a direct current at 10 mA/cm². The increase in voltage required for driving was evaluated by subtracting the initial driving voltage.

### Synthesis Example 1

13 ml of n-butyllithium (1.6 M hexane solution) was added dropwise at 0° C. to a mixed solution of 4.0 g of 1-bromo-3-chlorobenzene and 30 ml of tetrahydrofuran under a nitrogen flow. The mixture was stirred at 0° C. for 1 hour, and then added dropwise at 0° C. to a mixed liquid of 4.5 g of 2-phenyl-1,10-phenanthroline and 30 ml of tetrahydrofuran. After cooling to room temperature, the reaction solution was extracted with dichloromethane, and the extract was evaporated, leaving 100 ml of the solvent. 10.0 g of manganese dioxide was added to the resulting solution, and the mixture was stirred at room temperature for 4 hours. Then, magnesium sulfate was added, and the mixture was filtered, and the solvent was removed by evaporation. The resulting solid was purified by silica gel column chromatography, and the solvent was removed by evaporation, and the resulting solid was vacuum-dried to obtain 6.0 g of an intermediate.

Next, a mixed solution of 3.0 g of this intermediate, 3.7 g of a boronic acid ester represented by the following formula (15), 160 mg of dichlorobis(triphenylphosphine) palladium (II), 7 ml of a 1.5 M aqueous tripotassium phosphate solution, and 80 ml of 1,4-dioxane was heated and stirred under reflux for 7 hours under a nitrogen flow. After cooling to room temperature, water was added, filtration was performed, and the residue was washed with methanol and vacuum dried. The resulting solid was subjected to removal of a catalyst using activated carbon, and the solvent was removed by evaporation. The resulting solid was washed with toluene and methanol, and then vacuum-dried to obtain 4.4 g of a crude product of a phenanthroline derivative.

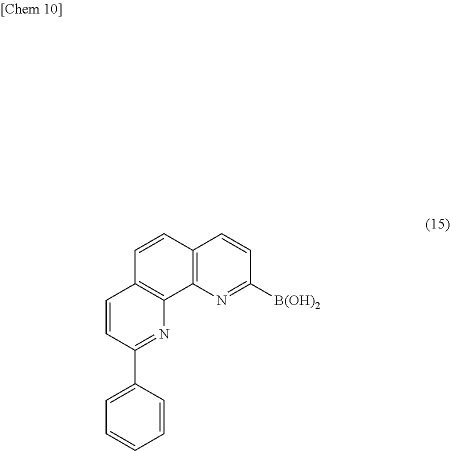 [SMILES: OB(O)c1ccc2ccc3ccc(-c4ccccc4)nc3c2n1]

The resulting crude product of the phenanthroline derivative was subjected to heated slurry washing with toluene, then recrystallized once with an anisole/toluene solution, and subjected to heated slurry washing with a tetrahydrofuran/methanol solution. The crystals were subjected to sublimation purification three times at 320° C. under a pressure of 1.0×10⁻³ Pa or less using an oil diffusion pump, thereby obtaining 3.0 g of the phenanthroline derivative of formula (3).

### Synthesis Example 2

2.8 g of a phenanthroline derivative of formula (4) was obtained in the same manner as in Synthesis Example 1, except that 4.5 g of 2-phenyl-1,10-phenanthroline was changed to 4.1 g of 1,10-phenanthroline.

### Synthesis Example 3

A phenanthroline derivative of formula (5) was obtained in the same manner as in Synthesis Example 2, except that 1,10-phenanthroline was changed to 2-nbutyl-1,10-phenanthroline.

### Synthesis Example 4

A mixed solution of 3.0 g of 2-phenyl-9-chloro-1,10-phenanthroline, 1.6 g of 3-chlorophenylboronic acid, 220 mg of dichlorobis(triphenylphosphine) palladium (II), 7 ml of a 1.5 M aqueous tripotassium phosphate solution, and 80 ml of 1,4-dioxane was heated and stirred under reflux for 7 hours under a nitrogen flow. After cooling to room temperature, water was added to precipitate a solid, filtration was performed, and the residue was washed with methanol and vacuum dried. The resulting solid was dissolved in tetrahydrofuran and subjected to removal of a catalyst using activated carbon, and the solvent was removed by evaporation. The resulting solid was washed with toluene and methanol, and then vacuum-dried.

Next, 3.0 g of the resulting solid, 3.1 g of bis(pinacolato)diboron, 70 mg of tris(dibenzylideneacetone) dipalladium (0), 80 mg of 2-dicyclohexylphosphino-2′,4′,6′-triisopropylbiphenyl, 2.4 g of potassium acetate, and DMF were dissolved in 80 mL and stirred at 120° C. for 24 hours under a nitrogen flow. After cooling to room temperature, water was added to precipitate a solid, filtration was performed, and the residue was further washed with methanol and vacuum dried. The resulting solid was dissolved in tetrahydrofuran and subjected to removal of a catalyst using activated carbon, and the solvent was removed by evaporation. The resulting solid was washed with toluene and methanol, and then vacuum-dried.

Next, 3.0 g of the resulting solid, 0.99 g of 2,9-dichloro-1,10-phenanthroline, 280 mg of dichlorobis(triphenylphosphine) palladium (II), and 2.2 g of potassium carbonate were dissolved in a mixed solvent of 80 ml of 1,4-dioxane and 20 mL of pure water, and the mixture was heated and stirred under reflux for 20 hours under a nitrogen flow. After cooling to room temperature, the solvent was removed by evaporation, and the resulting solid was recrystallized three times with a mixed solvent of tetrahydrofuran and methanol, followed by vacuum drying to obtain a phenanthroline derivative of formula (14).

### Example 1

(Step 1) to (Step 4) were carried out by a flow under the following conditions.
Syringe pump: Model YSP-301, high-performance high-pressure type, manufactured by YMC Co., Ltd.
Temperature: 10° C. to 30° C.
Flow channel diameter (inner diameter) between mixers: 1 mm
Material of flow channel between mixers: “Teflon” (registered trademark)
Mixer shape: T-shape
Mixer material: “Teflon” (registered trademark)
Mixer flow channel diameter: 1 mm.

### (Step 1)

A 0.2 M 1,3-dibromobenzene/tetrahydrofuran solution was fed from Server A into a flow path at a flow rate of 5.0 ml/min, a 0.2 M n-butyllithium/n-hexane solution was fed from Server B into the flow path at a flow rate of 4.6 ml/min, and the solutions were reacted in a first T-shaped mixer section. The flow rate of the reaction product was 9.6 ml/min.

### (Step 2)

After 2 seconds from the first T-shaped mixer section, a 0.2 M 2-phenyl-1,10-phenanthroline/tetrahydrofuran solution was fed from Server C into the flow path at a flow rate of 4.2 ml/min, and the solutions were reacted in a second T-shaped mixer section. The flow rate of the reaction product was 13.8 ml/min.

### (Step 3)

After 4 seconds from the second T-shaped mixer section, a 0.2 M n-butyllithium/n-hexane solution was fed from Server D into the flow path at a flow rate of 4.6 ml/min, and the solutions were reacted in a third T-shaped mixer section. The flow rate of the reaction product was 18.4 mL/min.

### (Step 4)

After 2 seconds from the third T-shaped mixer section, a mixed solution of a 0.2 M 2-phenyl-1,10-phenanthroline/tetrahydrofuran solution and a 0.032 mM 1,10-phenanthroline/tetrahydrofuran solution was fed from Server E into the flow path at a flow rate of 4.2 ml/min, and the solutions were reacted in a fourth T-shaped mixer section. After 14 seconds from the fourth T-shaped mixer section, the reaction product was collected in a flask. However, the collection of the reaction product was performed after 1 minute had passed since the pump started operating. Subsequently, (Step 5) and (Step 6) were carried out by the following method (batch).

### (Step 5)

The internal temperature of the flask was maintained at 0° C. to 5° C. in advance using ice water, and 50 ml of the solution of the reaction product obtained in (Step 5) was collected. Next, while maintaining the internal temperature of the flask at 0° C. to 5° C., the solution of the reaction product was stirred, 5 ml of H₂O was added to perform quenching. 50 ml of dichloromethane was then added to the quench solution to extract the reaction product.

To the solution of this extract, 50 ml of a dichloromethane solution in which 10 g of manganese dioxide was dispersed was added while maintaining the internal temperature of the flask at 30° C. or lower, followed by stirring for 15 minutes for oxidation. Next, the resulting oxide was filtered using a Kiriyama funnel (filter paper pore size: 4 μm), and the filtrate was concentrated and dried at a bath temperature of 40° C. using an evaporator to obtain 5.4 g of a mixed crude product of a phenanthroline derivative.

### (Step 6)

The mixed crude product of the phenanthroline derivative obtained in (Step 5) was subjected to heated slurry washing with toluene, then recrystallized once with an anisole/toluene solution, and subjected to heated slurry washing with a tetrahydrofuran/methanol solution. The crystals were subjected to sublimation purification once at 320° C. under a pressure of 1.0×10⁻³ Pa or less using an oil diffusion pump, thereby obtaining 4.4 g of a phenanthroline derivative mixture.

The resulting phenanthroline derivative mixture was evaluated by the above-described method, and the results are shown in Table 1, and the absorption spectrum obtained by analysis using HPLC is shown in FIG. 1.

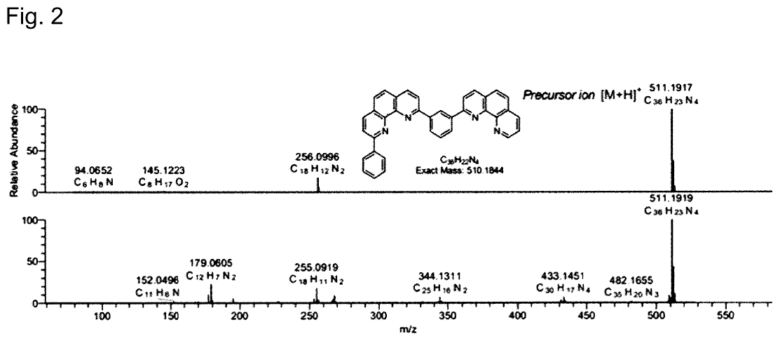

According to (2) the analysis of the structural formula by the mass spectrometry system, it was found that the compound having peaks at retention times of 12 minutes to 13 minutes was the compound of formula (3), that is, the phenanthroline derivative represented by formula (1), and the compound having peaks at retention times of 7 minutes to 8 minutes was the compound of formula (4), that is, the phenanthroline derivative represented by formula (A), based on the precursor ion values and product ion values in Table 3 and the mass spectrum shown in FIG. 2.

Of the total absorption intensity area 7, 607, 534 of all peaks with retention times of 7 to 20 minutes in FIG. 1, the absorption intensity area of the phenanthroline derivative represented by formula (1) was 7, 590, 720, and the absorption intensity area of the phenanthroline derivative represented by formula (4) was 1, 238. The absorption intensity area of the phenanthroline derivative represented by formula (4) was 0.016% with respect to the absorption intensity area of the phenanthroline derivative represented by formula (1).

The content of the phenanthroline derivative compound represented by formula (1) was 99.78% when calculated from the total absorption intensity area of all peaks with retention times of 7 minutes to 20 minutes and the absorption intensity area of the phenanthroline derivative represented by formula (1), and the content of the phenanthroline derivative represented by formula (4) was 0.016% when calculated from the absorption intensity area of the phenanthroline derivative represented by formula (4). In addition, when the light-emitting element of (3) was prepared and evaluated, the initial driving voltage was 8.5 (V) and the durability was 52, 1 hours. When (4) the durability evaluation of the light-emitting element during high-temperature driving was conducted, the durability was 29.1 hours, and when (5) the voltage evaluation of the voltage increase of high-temperature driving was conducted, the voltage increase was 0.30 V.

### Example 2

3.0 g of the phenanthroline derivative of formula (3) and 0.9 mg of the phenanthroline derivative of formula (4) were dissolved in a tetrahydrofuran/methanol solution, and sublimation purification was performed once at 320° C. under a pressure of 1.0×10⁻³ Pa or less using an oil diffusion pump, thereby obtaining 2.6 g of a mixture of the phenanthroline derivative of formula (3) and the phenanthroline derivative of formula (4). The resulting mixture of phenanthroline derivatives was evaluated in the same manner as in Example 1, and the results are shown in Table 1.

### Example 3

A mixture of phenanthroline derivatives was obtained in the same manner as in Example 2, except that 3.0 g of the phenanthroline derivative of formula (3) and 0.03 mg of the phenanthroline derivative of formula (4) were dissolved in a tetrahydrofuran/methanol solution. The resulting mixture of phenanthroline derivatives was evaluated in the same manner as in Example 1, and the results are shown in Table 1.

### Example 4

A mixture of phenanthroline derivatives was obtained in the same manner as in Example 3, except that the phenanthroline derivative represented by formula (5) was used instead of the phenanthroline derivative represented by formula (4). The resulting mixture of phenanthroline derivatives was evaluated in the same manner as in Example 1, and the results are shown in Table 1.

### Example 5

A mixture of phenanthroline derivatives was obtained in the same manner as in Example 2, except that 3.0 g of the phenanthroline derivative of formula (3) and 0.45 mg of the phenanthroline derivative of formula (5) were dissolved in a tetrahydrofuran/methanol solution. The resulting mixture of phenanthroline derivatives was evaluated in the same manner as in Example 1, and the results are shown in Table 1.

### Example 6

A mixture of phenanthroline derivatives was obtained in the same manner as in Example 2, except that 3.0 g of the phenanthroline derivative of formula (3) and 0.90 mg of the phenanthroline derivative of formula (5) were dissolved in a tetrahydrofuran/methanol solution. The resulting mixture of phenanthroline derivatives was evaluated in the same manner as in Example 1, and the results are shown in Table 1.

### Example 7

A mixture of phenanthroline derivatives was obtained in the same manner as in Example 2, except that 3.0 g of the phenanthroline derivative of formula (3) and 1.0 mg of the phenanthroline derivative of formula (5) were dissolved in a tetrahydrofuran/methanol solution. The resulting mixture of phenanthroline derivatives was evaluated in the same manner as in Example 1, and the results are shown in Table 1.

### Example 8

A mixture of phenanthroline derivatives was obtained in the same manner as in Example 2, except that 3.0 g of the phenanthroline derivative of formula (3) and 3.0 mg of the phenanthroline derivative of formula (5) were dissolved in a tetrahydrofuran/methanol solution. The resulting mixture of phenanthroline derivatives was evaluated in the same manner as in Example 1, and the results are shown in Table 1.

### Example 9

A mixture of phenanthroline derivatives was obtained in the same manner as in Example 2, except that 3.0 g of the phenanthroline derivative of formula (3) and 9.0 mg of the phenanthroline derivative of formula (5) were dissolved in a tetrahydrofuran/methanol solution. The resulting mixture of phenanthroline derivatives was evaluated in the same manner as in Example 1, and the results are shown in Table 1.

### Example 10

A mixture of phenanthroline derivatives was obtained in the same manner as in Example 2, except that 3.0 g of the phenanthroline derivative of formula (3) and 0.03 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 11

A mixture of phenanthroline derivatives was obtained in the same manner as in Example 2, except that 3.0 g of the phenanthroline derivative of formula (3) and 0.45 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 12

A mixture of phenanthroline derivatives was obtained in the same manner as in Example 2, except that 3.0 g of the phenanthroline derivative of formula (3) and 0.90 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 13

A mixture of phenanthroline derivatives was obtained in the same manner as in Example 2, except that 3.0 g of the phenanthroline derivative of formula (3) and 1.0 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 14

A mixture of phenanthroline derivatives was obtained in the same manner as in Example 2, except that 3.0 g of the phenanthroline derivative of formula (3) and 3.0 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 15

A mixture of phenanthroline derivatives was obtained in the same manner as in Example 2, except that 3.0 g of the phenanthroline derivative of formula (3) and 9.0 mg of the phenanthroline derivative of formula (14) were dissolved in a tetrahydrofuran/methanol solution. The resulting mixture of phenanthroline derivatives was evaluated in the same manner as in Example 1, and the results are shown in Table 1.

### Example 16

A mixture of phenanthroline derivatives was obtained in the same manner as in Example 2, except that 3.0 g of the phenanthroline derivative of formula (3), 0.03 mg of the phenanthroline derivative of formula (5), and 0.03 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Example 17

A mixture of phenanthroline derivatives was obtained in the same manner as in Example 2, except that 3.0 g of the phenanthroline derivative of formula (3), 0.45 mg of the phenanthroline derivative of formula (5), and 0.45 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Example 18

A mixture of phenanthroline derivatives was obtained in the same manner as in Example 2, except that 3.0 g of the phenanthroline derivative of formula (3), 4.5 mg of the phenanthroline derivative of formula (5), and 4.5 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Example 19

A mixture of phenanthroline derivatives was obtained in the same manner as in Example 2, except that 3.0 g of the phenanthroline derivative of formula (3), 0.03 mg of the phenanthroline derivative of formula (4), and 0.03 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Example 20

A mixture of phenanthroline derivatives was obtained in the same manner as in Example 2, except that 3.0 g of the phenanthroline derivative of formula (3), 0.45 mg of the phenanthroline derivative of formula (4), and 0.45 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Example 21

A mixture of phenanthroline derivatives was obtained in the same manner as in Example 2, except that 3.0 g of the phenanthroline derivative of formula (3), 4.5 mg of the phenanthroline derivative of formula (4), and 4.5 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

### Comparative Example 1

A phenanthroline derivative was obtained in the same manner as in Example 1, except that the solution fed from Server E in (Step 4) was set to only a 0.2 M 2-phenyl-1,10-phenanthroline/tetrahydrofuran solution. Evaluation results are shown in Table 1.

### Comparative Example 2

A mixture of phenanthroline derivatives was obtained in the same manner as in Example 2, except that 3.0 g of the phenanthroline derivative of formula (3) and 12.0 mg of the phenanthroline derivative of formula (4) were dissolved in a tetrahydrofuran/methanol solution. The resulting mixture of phenanthroline derivatives was evaluated in the same manner as in Example 1, and the results are shown in Table 2.

### Comparative Example 3

A mixture of phenanthroline derivatives was obtained in the same manner as in Example 2, except that 3.0 g of the phenanthroline derivative of formula (3) and 12.0 mg of the phenanthroline derivative of formula (5) were dissolved in a tetrahydrofuran/methanol solution. The resulting mixture of phenanthroline derivatives was evaluated in the same manner as in Example 1, and the results are shown in Table 2.

### Comparative Example 4

A mixture of phenanthroline derivatives was obtained in the same manner as in Example 2, except that 3.0 g of the phenanthroline derivative of formula (3) and 12.0 mg of the phenanthroline derivative of formula (14) were dissolved in a tetrahydrofuran/methanol solution. The resulting mixture of phenanthroline derivatives was evaluated in the same manner as in Example 1, and the results are shown in Table 2.

TABLE 1

| | | | Phenanthroline derivative(s) | | | | | | |
|---|---|---|---|---|---|---|---|---|---|
| | | | represented by formula (A) and/or | | | | | | |
| | Main component | | formula (B) | | | Initial | | Durability during | |
| | | Absorption | | Absorption | absorption | driving | | high-temperature | Increase |
| | | intensity | | intensity | intensity | voltage | Durability | driving | in voltage |
| | Compound | area | Compound | area | area ratio* | (V) | (Time) | (Time) | (V) |
| Example 1 | Formula (3) | 7,590,720 | Formula (4) | 1,238 | 0.016 | 8.5 | 52.1 | 29.1 | 0.30 |
| Example 2 | Formula (3) | 7,559,725 | Formula (4) | 2,272 | 0.030 | 8.5 | 51.8 | 29.5 | 0.30 |
| Example 3 | Formula (3) | 7,759,727 | Formula (4) | 72 | 0.001 | 8.4 | 52.5 | 30.3 | 0.30 |
| Example 4 | Formula (3) | 7,782,259 | Formula (5) | 77 | 0.001 | 8.4 | 52.4 | 41.1 | 0.20 |
| Example 5 | Formula (3) | 7,774,985 | Formula (5) | 1,310 | 0.017 | 8.4 | 52.3 | 43.4 | 0.20 |
| Example 6 | Formula (3) | 7,551,041 | Formula (5) | 2,199 | 0.029 | 8.5 | 51.7 | 42.8 | 0.20 |
| Example 7 | Formula (3) | 7,232,987 | Formula (5) | 2,458 | 0.034 | 8.6 | 52.9 | 38.5 | 0.20 |
| Example 8 | Formula (3) | 7,698,374 | Formula (5) | 7,487 | 0.097 | 8.6 | 53.5 | 37.0 | 0.20 |
| Example 9 | Formula (3) | 7,554,089 | Formula (5) | 22,587 | 0.299 | 8.6 | 52.0 | 35.5 | 0.20 |
| Example 10 | Formula (3) | 7,736,197 | Formula (14) | 77 | 0.001 | 8.5 | 54.5 | 44.4 | 0.10 |
| Example 11 | Formula (3) | 7,773,719 | Formula (14) | 1,168 | 0.015 | 8.4 | 55.6 | 44.4 | 0.10 |
| Example 12 | Formula (3) | 7,556,230 | Formula (14) | 2,199 | 0.029 | 8.4 | 55.1 | 45.6 | 0.10 |
| Example 13 | Formula (3) | 7,660,374 | Formula (14) | 2,668 | 0.035 | 8.5 | 54.0 | 39.5 | 0.10 |
| Example 14 | Formula (3) | 7,779,734 | Formula (14) | 7,678 | 0.099 | 8.5 | 53.6 | 37.1 | 0.10 |
| Example 15 | Formula (3) | 7,554,044 | Formula (14) | 22,592 | 0.299 | 8.6 | 52.6 | 35.8 | 0.10 |

*(Absorption intensity area of phenanthroline derivative(s) represented by formula (A) and/or formula (B))/(absorption intensity area of phenanthroline derivative represented by formula (1)) × 100

TABLE 2

| | | | Phenanthroline derivative(s) | | | | | | |
|---|---|---|---|---|---|---|---|---|---|
| | | | represented by formula (A) and/or | | | | | | |
| | | | formula (B) | | | | | | |
| | Main component | | | | absorption | Initial | | Durability during | |
| | | Absorption | | Absorption | intensity | driving | | high-temperature | Increase |
| | | intensity | | intensity | area | voltage | Durability | driving | in voltage |
| | Compound | area | Compound | area | ratio* | (V) | (Time) | (Time) | (V) |
| Example 16 | Formula (3) | 7,780,099 | Formula (5) + | 154 | 0.002 | 8.5 | 55.0 | 50.3 | 0.01 |
| | | | formula (14) | | | | | | |
| Example 17 | Formula (3) | 7,555,901 | Formula (5) + | 2,267 | 0.030 | 8.4 | 53.7 | 50.5 | 0.01 |
| | | | formula (14) | | | | | | |
| Example 18 | Formula (3) | 7,553,555 | Formula (5) + | 22,342 | 0.296 | 8.4 | 53.2 | 47.0 | 0.01 |
| | | | formula (14) | | | | | | |
| Example 19 | Formula (3) | 7,779,678 | Formula (4) + | 156 | 0.002 | 8.5 | 55.7 | 50.7 | 0.10 |
| | | | formula (14) | | | | | | |
| Example 20 | Formula (3) | 7,551,890 | Formula (4) + | 2,287 | 0.030 | 8.4 | 53.1 | 50.8 | 0.10 |
| | | | formula (14) | | | | | | |
| Example 21 | Formula (3) | 7,558,179 | Formula (4) + | 22,288 | 0.292 | 8.4 | 53.1 | 47.5 | 0.10 |
| | | | formula (14) | | | | | | |
| Comparative | Formula (3) | 7,730,007 | No peak | | | 8.3 | 50.3 | 25.1 | 0.50 |
| Example 1 | | | | | | | | | |
| Comparative | Formula (3) | 7,689,759 | Formula (4) | 30,877 | 0.402 | 9.0 | 51.6 | 30.1 | 0.30 |
| Example 2 | | | | | | | | | |
| Comparative | Formula (3) | 7,688,211 | Formula (5) | 31,231 | 0.406 | 9.1 | 52.3 | 31.2 | 0.20 |
| Example 3 | | | | | | | | | |
| Comparative | Formula (3) | 7,693,629 | Formula (14) | 30,960 | 0.402 | 9.1 | 54.0 | 41.6 | 0.10 |
| Example 4 | | | | | | | | | |

*(Absorption intensity area of phenanthroline derivative(s) represented by formula (A) and/or formula (B))/(absorption intensity area of phenanthroline derivative represented by formula (1)) × 100

TABLE 3

| | Molecular | Precursor | Product |
|---|---|---|---|
| Compound | weight | ion (m/z) | ion (m/z) |
| Formula (4) | 510.1844 | 511.1917 | 511.1919 |
| Formula (5) | 566.2470 | 567.2445 | 567.2446 |

From the results of Tables 1 and 2, it was found that when the absorption intensity area of the phenanthroline derivative(s) represented by formula (4) and/or formula (5) and/or formula (14), that is, the phenanthroline derivative(s) represented by formula (A) and/or the following formula (B), analyzed by high-performance liquid chromatography, is 0.001% to 0.300% with respect to the absorption intensity area of the phenanthroline derivative represented by formula (1), an element having excellent durability can be obtained.

1. A phenanthroline derivative having a main component represented by the following formula (1), wherein, when analyzed by high-performance liquid chromatography, the absorption intensity area of a phenanthroline derivative represented by the following formula (A) and/or the following formula (B) is 0.001% to 0.300% with respect to the absorption intensity area of the phenanthroline derivative represented by the following formula (1), 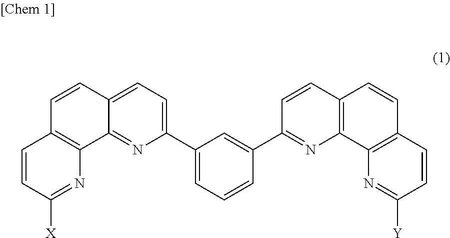
(where the above X and Y each independently represent a substituted or unsubstituted aryl group; and X and Y may be the same or different), 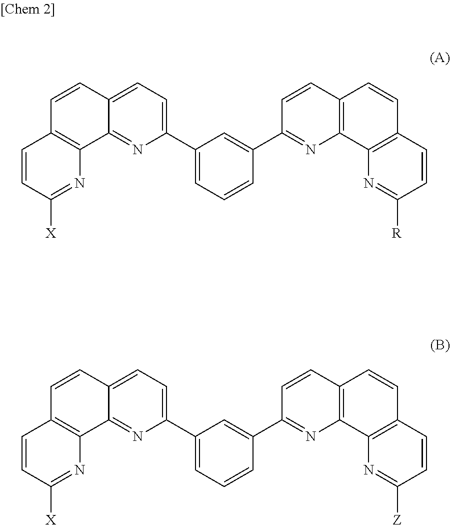
(where X represents a substituted or unsubstituted aryl group; R represents any of a hydrogen atom, a halogen atom, a cyano group, a substituted or unsubstituted alkyl group having 1 to 9 carbon atoms, a cycloalkyl group, a heterocyclic group, an alkenyl group, a cycloalkenyl group, an alkynyl group, or an alkoxy group; and Z is a substituted aryl group, provided that the phenanthroline derivative represented by general formula (B) has a structure different from that of the phenanthroline derivative represented by general formula (1)).

2. The phenanthroline derivative according to claim 1, wherein the phenanthroline derivative represented by the formula (1) is a phenanthroline derivative represented by the following formula (3)

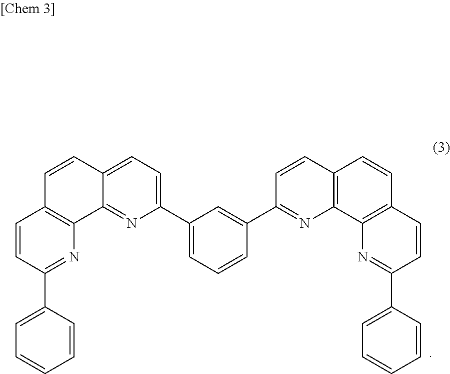 [SMILES: c1ccc(-c2ccc3ccc4ccc(-c5cccc(-c6ccc7ccc8ccc(-c9ccccc9)nc8c7n6)c5)nc4c3n2)cc1]

3. The phenanthroline derivative according to claim 1, wherein the phenanthroline derivative represented by the formula (A) and/or the formula (B) is one or more compounds selected from the group consisting of the following formulas (4), (5), and (14)

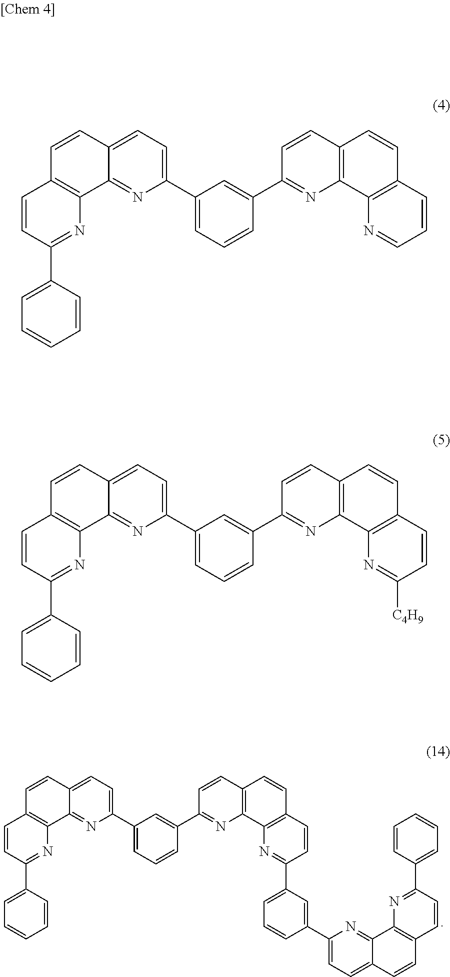

4. The phenanthroline derivative according to claim 3, wherein the phenanthroline derivative represented by the formula (A) and/or the formula (B) is two or more compounds selected from the group consisting of the formulas (4), (5), and (14).

5. The phenanthroline derivative according to claim 4, wherein the phenanthroline derivative represented by the formula (A) and/or the formula (B) includes at least both the compound represented by the formula (5) and the compound represented by the formula (14).

6. The phenanthroline derivative according to claim 1, wherein, when analyzed by the high-performance liquid chromatography, the absorption intensity area of a phenanthroline derivative represented by the formula (A) and/or the formula (B) is 0.001% to 0.030% with respect to the absorption intensity area of the phenanthroline derivative represented by the formula (1).

7. The phenanthroline derivative according to claim 1, wherein the conditions for analysis by the high-performance liquid chromatography are as follows:
Apparatus: Nexera lite system (LC-40 series) manufactured by Shimadzu Corporation
Detector: Photodiode array detector (SPD-M40) manufactured by Shimadzu Corporation
Column: A reversed-phase column packed with a filler of high-purity spherical silica gel particles chemically bonded with octyl groups
Theoretical plate number: 105,000±10,000 (N/m)
Column temperature: 45° C.
Flow rate: 1.0 mL/min
Injection amount: 10 UL
Measurement sample concentration: 5.0 mg/40 ml of tetrahydrofuran
Mobile phase: Solution A—an aqueous phosphoric acid solution having a concentration of 0.1 weight %; Solution B—an acetonitrile/tetrahydrofuran mixed solution (weight ratio 80/20)
Liquid feeding conditions: At first, a volume ratio of solution A/solution B is 55/45; after a retention time of 25 minutes, only solution B; with a linear gradient for the first 25 minutes
Analysis software: Lab Solutions manufactured by Shimadzu Corporation
Measurement wavelength: 254 nm
Detection area of phenanthroline derivative: 6,000,000 or more
Minimum detectable area of compound: 60
Width (W): 1 sec
Slope(S): 1,000 ρV/min
Drift (D): 300 μV/min
T.DBL (T): 1,000 min.

8. A method for producing a phenanthroline derivative comprising, in this order: (Step 1) a step of reacting a 1,3-dihalogenated aromatic compound with an organolithium reagent; (Step 2) a step of reacting a lithiated reaction product of Step 1 with a phenanthroline derivative of the following formula (6); (Step 3) a step of reacting the reaction product of Step 2 with an organolithium reagent; (Step 4) a step of reacting the lithiated reaction product of Step 3 with a phenanthroline derivative represented by the following formula (7) and a phenanthroline derivative represented by the following formula (8); (Step 5) a step of oxidizing the reaction product of Step 4 to obtain a crude product of the phenanthroline derivative; and (Step 6) a step of recrystallizing and further sublimating the crude product of the phenanthroline derivative of Step 5, 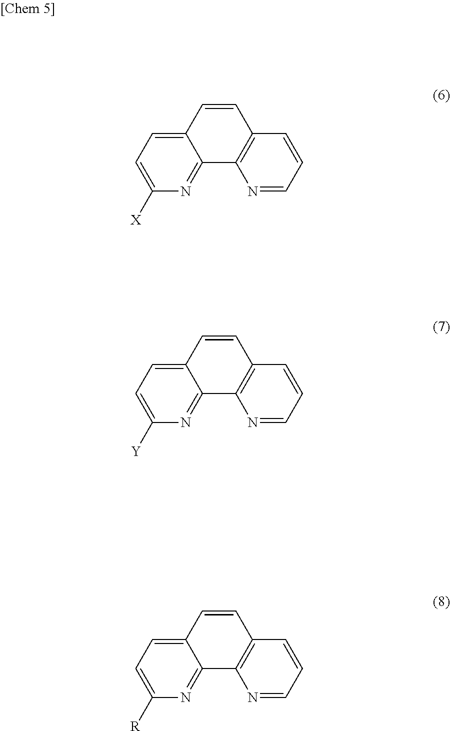
(where the above X and Y each independently represent a substituted or unsubstituted aryl group; and R represents any of a hydrogen atom, a halogen atom, a cyano group, a substituted or unsubstituted alkyl group having 1 to 9 carbon atoms, a cycloalkyl group, a heterocyclic group, an alkenyl group, a cycloalkenyl group, an alkynyl group, or an alkoxy group).

9. A light-emitting element comprising: the phenanthroline derivative according to claim 1.

10. A display device comprising: the light-emitting element according to claim 9.
